(19) United States
(12) Patent Application Publication
(10) Pub. No.: US 2006/0124246 A1
(43) Pub. Date: Jun. 15, 2006

(54) Moistening fluids that destroy and/or inhibit the growth of biological organisms

(75) Inventors: Maureen A. Spisinski, North Branford, CT (US); Richard A. Bernard, Norwalk, CT (US); William E. Ryan JR., Monroe, CT (US)

Correspondence Address: PITNEY BOWES INC.;35 WATERVIEW DRIVE, P.O. BOX 3000, MSC 26-22, SHELTON, CT, 06484-8000, US

(73) Assignee: Pitney Bowes Incorporated, Stamford, CT (US)

(21) Appl. No.: 11/011,268

(22) Filed: Dec. 14, 2004

(51) Int. Cl. B43M 3/00; A61L 2/02; B01J 19/00

(52) U.S. Cl. 156/441.5; 422/1; 422/243; 422/291

(57) ABSTRACT

Moistening fluids are disclosed that are capable of being used in mail processing machines and systems. Mail processing equipment can automatically feed and moisten envelopes at slow to very high speeds of 30 inches per second. The moistening system becomes contaminated with paper dust, talc, and common envelope adhesives. The moistening fluids kill many types of bacteria, fungi, and inhibit the growth of other types of bacteria, fungi, and algae. The moistening fluids of this invention are safe for use in an office environment, i.e., they are non-toxic and non-flammable and may be transported by common carriers without any safety precautions. The moistening fluid contains: detergent, biocide, alcohol, dye and distilled water.

### FIELD OF THE INVENTION

The invention relates generally to moistening fluids and more particularly to moistening fluids that destroys and/or inhibit the growth of biological organisms.

### BACKGROUND OF THE INVENTION

In mail processing systems, moistening devices are used to wet the flap of an envelope in preparation for sealing the envelope or moistening a tape for sealing objects, or adhering labels. Conventionally, flap wetting is accomplished by feeding the envelopes flaps past a moisture applicator such as a brush, a wick, or other moist surface, allowing the moist surface to come into contact with the water-moistening adhesive on the envelope flap. The moisture causes the adhesive to soften and to become sticky. The moistened envelopes' flaps are closed and the adhesive is pressed between the flap and the body of the envelope to form a seal. The envelope is then either ejected into a stacker, or passed on to another part of the mail processing system for further processing.

Envelope flap moistening devices generally fall into two categories, contact and non-contact moistening devices. Contact systems generally deposit moisture onto an envelope flap by contact with a wetted substrate or narrow slotted device that allows fluid to flow, based upon the capillary action of the slotted device's contact with the flap. Non-contact systems generally spray moisture onto the envelope flap. In non-contact flap moistening systems, envelope flap moistening has been performed with a nozzle and the aid of a pumping system. In the aforementioned systems the moistening fluid is stored in a reservoir or remains in internal tubing, where certain types of bacteria, fungi, and algae have an opportunity to grow.

The bacteria, fungi and algae have a natural source of food because the envelope adhesive generally contains dextrin i.e., a corn and/or potato starch.

The following microorganisms were found in representative samples taken from moistening systems: Sphingomonas paucimobilis (bacteria); Geotrichum species (fungus); Yeasts; Pseudomonas stutzeri (bacteria); Fusarium species (mold); Aspergillus niger (mold); Acinetobacter species (bacteria); Blue green algae; Caulobacter species (bacteria); Pseudomonas aeruginosa (bacteria): Pseudomonas fluorescens (bacteria); Brevendimonas species (bacteria); Flavomonas species (bacteria); Cladosporium species (fungus); Oididendron species (fungus) Penicillium species (mold).

A disadvantage of current moistening devices is that they use moistening fluids that are conducive to the growth of bacteria, fungi, and algae.

Another disadvantage of current moistening devices is that sometimes-significant amounts of bacteria, fungi, and algae grow before the moistening fluid is completely used, and due to the inappropriate selection of a biocide or an insufficient concentration of the biocide, it is not adequate to stop growth.

A further disadvantage of current moistening devices is that the growth of bacteria, fungi, and algae may result in the inconsistent wicking of the substrate causing it to become unevenly saturated, which results in decreased moistening and/or clogging of the filters and tubing in moistening systems.

A further disadvantage of current systems is that the excessive growth of bacteria, fungi, and algae results in the production of unpleasant odors.

### SUMMARY OF THE INVENTION

This invention overcomes the disadvantages of the prior art by providing moistening fluids that destroys many types of bacteria, fungi, and inhibits the growth of other types of bacteria, fungi, and algae. The moistening fluids of this invention are safe for use in an office environment, i.e., they are non-toxic and may be transported by common carriers without any safety precautions. The moistening fluids may be used in mailing systems to seal envelope flaps, adhere labels to mail pieces, i.e., letters, flats, or packages. They may also be used to adhere labels and tapes to objects. The moistening fluids may also be placed in a device or dispenser that is a stand-alone container.

An advantage of this invention is that the surface tension of the moistening fluid is within the range of 28.5-35.1 dynes/cm. Disinfectants are usually solutions of low surface tension. This allows them to spread out on the cell walls of bacteria and disrupt them.

Properties of the moistening fluids under consideration are conductivity, alkalinity/acidity, wicking, surface tension, corrosion resistance, moistening sealing weight viscosity and zone of inhibition.

“Conductivity is the ability of a material to conduct electric current. Since the charge on ions in solution facilitates the conductance of electrical current, the conductivity of a solution is proportional to its ion concentration.” Thus, the conductivity is an important physical parameter in the preparation of the moistening fluids described herein, since certain moistening systems have ion detection devices which signal the user of the system that the system is low or out of moistening solution.

pH is a value taken to represent the acidity or alkalinity of an aqueous solution; it is defined as the logarithm of the reciprocal of hydrogen-ion concentration of a solution.

Alkalinity is the measurement of pH value above 7 and acidity is the measurement of a pH value below 7.

The pH value is important, because it demonstrates the moistening fluid's ability to be handled safely and operate safely in most systems.

The ability to destroy and/or inhibit the growth of certain types of bacteria, fungi, and algae is improved by the addition of detergents, biocides, and alcohols at specific ratios.

### DETAILED DESCRIPTION OF THE PREFERRED EMBODIMENT

The formulation of the moistening fluids of the instant invention is capable of being used in mail processing machines and systems. Composition of the moistening fluids, in accordance with the invention comprises detergent, biocide, alcohol, dye and water.

The general composition of the moistening fluids is as follows:

| INGREDIENT | (WT. %) |
|---|---|
| Detergent | Range 5.75-14.95 |
| Biocide | Range 0.005-0.107 |
| Alcohol | Range 3.86-6.22 |
| Dye | Range 0.00-0.000015 |
| Distilled Water | Range 78.77-90.33 |

The following detergents were used in the examples described herein. Alkylether hydroxypropyl sultaine manufactured by Burlington Chemical of Post Office Box 111, 615 Huffman Mill Road, Burlington, N.C.

The following biocides were used in the examples described herein:
1. Alkyl(C₁₄ 50%, C₁₆ 10%, C₁₂ 40%) Dimethyl Benzyl Ammonium Chloride manufactured by Lonza of 17-17 Route 208, Fair Lawn, N.J.
2. 5-Chloro-2-Methyl-4-Isothioazolin-3-one manufactured by Rohm and Haas Company of 100 Independence Mall West, Philadelphia, Pa.
3. Glycine, N-(hydroxymethyl)-monosodium salt manufactured by ISP Chemicals Inc. of 1361 Alps Road, Wayne, N.J., 07470.
4. 1,2-Benzisothiazoline-3-one manufactured by Avecia, Inc. of P.O. Box 15457, Wilmington, Del.

Alcohols
1. 2-Propanol
2. Ethanol (denatured) composed of ethyl alcohol, 95.27%; Methyl Isobutyl ketone 1.0%; Ethyl Acetate, 1.0%; Hydrocarbon, 1.0%; water≦0.5%
3. n-Propanol
4. 1-Butanol

Dye
1. FD &C #1 Blue Dye—(Food Drug & Cosmetic #1 Blue Dye)

Water
1. Distilled Water

To determine the efficacy of the moistening fluids to inhibit and/or kill bacteria, fungi, and algae, a Zone of Inhibition test was conducted. The procedure for Zone of Inhibition testing is as follows:

Equipment
Sterile Tryptic Soy Agar plates (TSA)
Sample contaminants in sterile diluent solution manufactured by Fabriqué au Canada par Starplex Scientific Inc., 50 Steinway Blvd., Etobicoke, Ontario, Canada
The sample contaminant used contained one or more of the following organisms:
   1. Geotrichum species (fungus)
   2. Yeasts
   3. Pseudomonas stutzeri (bacteria)
   4. Fusarium species (mold)
   5. Aspergillus niger (mold)
   6. Acinetobacter species (bacteria)
   7. Blue green algae
   8. Caulobacter species (bacteria)
   9. Pseudomonas aeruginosa (bacteria)
   10. Pseudomonas fluorescens (bacteria)
   11. Brevendimonas species (bacteria)
   12. Flavomonas species (bacteria)
   13. Cladosporium species (fungus)
   14. Oididendron species (fungus)
   15. Sphingomonas paucimobilis (bacteria)
   16. Penicillium species (mold
16 mm discs cut from Whatman 41 filter paper
Millipore 5.0 um 25 mm discs
Pall 45 um membrane filters
Metric ruler
Moistening fluid (Examples 1-21)
Distilled water
Incubating oven set for 35° C.

Procedure
Swab sterile Tryptic Soy Agar plates (TSA) with solution of sample contaminant.
Place one disc, or membrane filter, which has been saturated with the moistening fluid on center of plate.
Saturate one disc in distilled water and utilize as a control
Incubate overnight in 35° C. incubating oven.
At 24-hour intervals, measure the linear distance that has been cleared of microbial growth and record from the filter disc's circumference to the outer perimeter of the cleared area. This is the Zone of inhibition.
Take measurements each 24-hour period for three (3) days.

To determine the ability of the moistening fluids to seal envelopes in mailing systems envelope, sealing tests were performed.

Equipment: #10 envelopes with water moisturizing adhesive
Moistening fluids, as described
Mettler PE 3600 balance
Moistening Mailing system(s)
Deionized and/or Distilled water
Procedure: One hundred milliliters (mis) of moistening fluid to be tested was added to the moistening fluid reservoir of the moistening mailing system.
Fifty (50) #10 envelopes with moisturizing adhesive were weighed dry.
These same fifty (50) envelopes were then sent through the moistening mailing system, in order to wet and seal the envelopes.
The 50 envelopes were re-weighed immediately to determine the amount of gross weight gain of the envelopes.
The mailing system was rinsed with either distilled or deionized water, in order to remove traces of the previously run moistening fluid. This was accomplished by running twenty-five (25) envelopes through the system with either distilled or deionized water.
This procedure was conducted a minimum of two times, for each example in order to determine the average weight gain of fifty (50) envelopes of each moistening fluid.
A baseline average weight gain was determined by averaging the gross weight gain for each group of fifty (50) envelopes tested, for each example.

To determine the speed of wicking, i.e., the ability of a fluid to be drawn up the fibers, of a brush of a moistening system, wicking tests were performed.
Equipment: Esterlon (polyester) F984031, unbaked brushes, flagged 1 pass 1.5 seconds/side
   Ring stand equipped with two side arm clamps
   Lap/split timer by Fisher Scientific of Hanover Park, Ill.
   Whatman 11.0 cm 40 Ashless filter paper circles
   Moistening fluids as described
   Sterile empty petri dishes
Procedure: Small ring stand with two side arm clamps was assembled in hood
   One side arm clamp was used to hold the Esterlon brush in an upright position
   The second side arm clamp held the filter paper circle fixed on the brush's fibrous end
   Sterile petri dish was filled with the appropriately labeled moistening fluids
   At the point where the lower end of the brush came in contact with the moistening fluids, the timer was activated to measure the time it took the moistening fluids to travel up the bristles of the brush and moisten the filter paper.
   Once moisture was visible on the filter paper, the timer was stopped and the time recorded.

To determine the ability of the moistening fluids to inhibit and/or destroy the growth of specific bacteria, fungi and algae, in mailing systems, the moistening fluid was subjected to Challenge Testing (Modified American Society For Testing Materials (ASTM) D-2574), with the following organisms:

| | |
|---|---|
| Acinetobacter sp. | Sacchromyces cereviseae |
| Penicillium sp. | Candida albicans |
| Cladosporium sp. | Pseudomonas aeruginosa |
| Geotrichum sp. | Escherichia coli |
| Caulobacter sp. | Aspergillus niger |
| Mixed Algae Pond Collection | Controls of Distilled Water and |
| | Tap Water |

The Challenge Test consists of a 7-day study for the above mentioned microorganisms in which each microorganism is inoculated into an aliquot of moistening fluid and subsequently tested as to the viability of each organism, after specific increments of contact time.

Material: Five (5) test tubes with 9.0 ml. of sterile diluent solution for each microorganism tested; American Type Culture Collection (ATCC) cultures of specified type; Thirty-two (32) Sterile Tryptic Soy Agar plates, sterile disposable 1 ml. pipettes; pipette aids;

Procedure: Obtain pure stock culture of each organism from accredited vendor, such as American Type Culture Collection (ATCC). Perform serial dilutions to determine actual inoculum microbial count for each organism.
Serial dilutions performed by
   Take five test tubes for each organism, each containing 9.0 ml. of sterile diluent solution, and label with appropriate dilution factor (i.e. 1:10; 1:100, 1:1000, 1:10,000 etc.)
   Take 1 ml. of the original inoculum and inoculate the first tube of 9.0 ml. of sterile diluent solution. Mix well and plate 0.1 ml. of the diluent solution/microbial culture to each of two (2) sterile Tryptic Soy Agar plates. Draw a 1 ml. aliquot from the first dilution (1:10) and inoculate the second dilution (1:100) Mix well and plate one (1) ml. of the dilution to each of two (2) sterile Tryptic Soy Agar plates. Continue to transfer the same amount (1 ml.) to each successive dilution and mix well. Continue to plate 1 ml. of each dilution to two (2) sterile Tryptic Soy Agar plates. Incubate at optimal temperature for each organism. After 48 hours, count the colony forming units (CFU) on each plate, at each dilution and record. The number of microorganisms in the original inoculum equals the averaged number of colony forming units (cfus) from the duplicate plates times the dilution of the sample.
Once the number of bacteria/ml for each microorganism being tested has been determined:
   Prepare a test tube for each tested microorganism with 9.9 mls. of the moistening fluid.
   Inoculate 0.1 mis. of the microorganism to be tested into the 9.9 mls. of moistening fluid and mix well.
   Immediately draw one ml. (1 ml.) of the freshly inoculated moistening fluid and plate onto a sterile Tryptic Soy Agar plate. Continue by plating the duplicate plate. Swirl plate to ensure the even distribution of the fluid. Mark the duplicate plates with the organism's identification and designate as Time 0.

Allow the vials with the moistening fluid/microorganism inoculum to remain undisturbed in a biological safety cabinet for 24 hours.
At the 24 hour mark, mix each vial well and draw, one (1) ml. of the moistening fluid/microorganism inoculum and plate onto a sterile Tryptic Soy Agar plate. Plate duplicate plate. Label as Time 24
Repeat the procedure at the 48 hour and 72 hour period of contact, and label as Time 48 and Time 72, respectively.
Incubate all of the plates at 27° C. for seven days.
Read the countable plates (those plates with cfus between 30-300)
Record the number of cfus/ml for each plate.
Determine the log reduction achieved within 24 hours of contact time, 48 hours of contact tine and 72 hours and 7 days of contact time, for each microorganism tested.

Log Reduction Explanation:

“Log” stands for logarithm, which is the exponent of 10. For example, log² or 10×10 or 100 for a 10-fold or one decimal or 90% reduction in numbers of recoverable bacteria in a test food vehicle. And 1 log reduction would reduce the number of bacteria 90%. The 5 log refers to 10 to the 5^{th} power or reduction in the number of microorganisms by 100,000-fold. For example, a product containing 100,000 pertinent microorganisms, a 5-log reduction would reduce the number or pertinent micro-organisms by 99.999%.

Two Practical Ways of Looking at 5-Log Reduction:

1. Reduction of 100,000 bad microorganisms in one contaminated serving to 1 bad microorganism in a serving.

2. Reduction of 100,000 contaminated servings to 1 contaminated serving.

Log Reduction Chart

| | % Reduction of |
|---|---|
| Log Reduction | Bacteria |
| 1 | 90 |
| 2 | 99 |
| 3 | 99.9 |
| 4 | 99.99 |
| 5 | 99.999 |

The surface tension of a liquid is the attractive force exerted by the molecules below the surface upon those at the surface/air interface. An internal pressure is thus created, which tends to restrain the liquid from flowing. Water is typically around 73 dynes/cm at 20° C.

The viscosity is the internal resistance to flow exhibited by a fluid, the ratio of shearing stress to rate of shear. The unit of viscosity is poise which equals 100 centipoise.

The following examples are exemplary of the invention and should not be considered as limiting.

### EXAMPLE 1

Composition

| Detergent (Alkylether hydroxypropyl sultaine) | | 11.500 | wt % |
|---|---|---|---|
| Biocide | Alkyl (C₁₄ 50%; C₁₆ 10%, C₁₂ 40%) | 0.023 | wt % |
| | Dimethyl Benzyl Ammonium Chloride | | |

| | | |
|---|---|---|
| 2-Propanol | 4.950 | wt % |
| Dye | 0.000015 | wt % |
| Distilled Water | 83.526985 | wt % |

Properties of Example 1

The pH of the moistening fluid is 6.3

The conductivity of the moistening fluid is 5.29 mmhos

The surface tension of this moistening fluid is 31.0 dynes/cm

Zone of inhibition 2 mm with a 16 mm saturated disc at 24 hours.

Zone of inhibition 2.5 mm with a 16 mm saturated disc at 48 hours.

Zone of inhibition 2.5 mm with a 16 mm saturated disc at 72 hours.

Gross Weight Gain—Sealing Test 1.235 g.

Wicking Test Times:

| | |
|---|---|
| Measurement No. 1: | 0.78 seconds |
| Measurement No. 2: | 0.99 seconds |
| Measurement No. 3: | 0.99 seconds |
| Average Measurement: | 0.92 seconds |

Viscosity—1.41 cps.

Performance Of Example 1
1. The moistening fluid's biocidal capability was at an acceptable level with no regrowth visible in the zone of inhibition.
2. The moistening fluid's conductivity was sufficiently high to be acceptable for use in mailing systems with ion detecting moistening fluids sensors.
3. The results of the sealing test were satisfactory, in that all the envelopes sealed.
4. The viscosity, surface tension and pH were within acceptable range.

### EXAMPLE 2

Composition

| Detergent (Alkylether hydroxypropyl sultaine) | | 8.050 | wt % |
|---|---|---|---|
| Biocide | Alkyl (C₁₄ 50%; C₁₆ 10%, C₁₂ 40%) | 0.023 | wt % |
| | Dimethyl Benzyl Ammonium Chloride | | |

| | | |
|---|---|---|
| 2-Propanol | 4.950 | wt % |
| Dye | 0.000015 | wt % |
| Distilled Water | 86.976985 | wt % |

Properties of Example 2

The pH of the moistening fluid is 6.1.

The conductivity of the moistening fluid is 4.08 millimhos.

The surface tension of the moistening fluid is 31.6 dynes/cm.

Zone of inhibition 4.75 mm with a 16 mm saturated disc at 24 hours.

Zone of inhibition 5.0 mm with a 16 mm saturated disc at 48 hours.

Zone of inhibition 5.0 mm with a 16 mm saturated disc at 72 hours.

Gross Weight Gain—Sealing Test 1.310 g.

Wicking Test Times:

| | |
|---|---|
| Measurement No. 1: | 0.75 seconds |
| Measurement No. 2: | 1.00 seconds |
| Measurement No. 3: | 0.95 seconds |
| Average Measurement: | 0.90 seconds |

Viscosity—1.29 cps.

Challenge Test

Log Reductions

| Organism | Zero Time | 24 Hours | 48 Hours | 72 Hours | 7 Days |
|---|---|---|---|---|---|
| Caulobacter | >3.7 | >3.7 | >3.7 | >3.7 | >4.7 |
| Acinetobacter | >3.8 | >3.8 | >3.8 | >3.8 | >4.8 |
| Penicillium | >3.5 | >3.5 | >3.5 | >3.5 | >4.5 |
| Cladosporium | >3.7 | >3.7 | >3.7 | >3.7 | >4.7 |
| Geotrichum | >3.5 | >3.5 | >3.5 | >3.5 | >4.5 |
| Algae | — | >4.3 | >4.3 | >4.3 | >4.3 |
| Ecoli | >3.1 | >3.1 | >3.1 | >3.1 | >4.1 |
| Pseudomonas | >3.5 | >3.5 | >3.5 | >3.5 | >4.4 |
| Candida | 3.2 | >3.2 | >3.2 | >3.2 | >4.2 |
| Aspergillus | 0.78 | >3.0 | >3.0 | >3.0 | >4.0 |
| Sacchromyces | 3 | >3.0 | >3.0 | >3.0 | >4.0 |
| cereviseae | | | | | |

Performance of Example 2
1. The moistening fluid's biocidal capability was at an acceptable level with no regrowth visible in the zone of inhibition.
2. The moistening fluid's conductivity was sufficiently high to be acceptable for use in mailing systems with ion detecting moistening fluids sensors.
3. The results of the sealing test were satisfactory, in that all the envelopes sealed.
4. The challenge test results indicated acceptable immediate and sustained microbial log reduction of target organisms.
5. The viscosity, surface tension and pH were within a acceptable range.

### EXAMPLE 3

Composition

| Detergent (Alkylether hydroxypropyl sultaine) | | 5.750 | wt % |
|---|---|---|---|
| Biocide | Alkyl (C₁₄ 50%; C₁₆ 10%, C₁₂ 40%) | 0.023 | wt % |
| | Dimethyl Benzyl Ammonium Chloride | | |

| | | |
|---|---|---|
| 2-Propanol | 4.950 | wt % |
| Dye | 0.000015 | wt % |
| Distilled Water | 89.276985 | wt % |

Properties of Example 3

The pH of the moistening fluid is 6.1.

The conductivity of the moistening fluid is 3.06 mmhos

The surface tension of the moistening fluid is 33.7 dynes/cm.

Zone of Inhibition 3.0 mm with a 16 mm saturated disc at 24 hours.

Zone of Inhibition 3.0 mm with a 16 mm saturated disc at 48 hours.

Zone of Inhibition 3.0 mm with a 16 mm saturated disc at 72 hours.

Gross Weight Gain—Sealing Test=0.890 g.

Wicking Test Times

| | |
|---|---|
| Measurement No. 1: | 1.79 seconds |
| Measurement No. 2: | 1.50 seconds |
| Measurement No. 3: | 1.03 seconds |
| Average Measurement: | 1.44 seconds |

Viscosity—1.32 cps.

Performance of this Example
1. The moistening fluid's biocidal capability was at an acceptable level with no regrowth visible in the zone of inhibition.
2. The moistening fluid's conductivity was sufficiently high to be acceptable for use in mailing systems with ion detecting moistening fluids sensors.
3. The results of the sealing test were satisfactory, in that all the envelopes sealed.
4. The viscosity, surface tension and pH were within an acceptable range.

### EXAMPLE 4

Composition

| Detergent (Alkylether hydroxypropyl sultaine) | | 14.950 | wt % |
|---|---|---|---|
| Biocide | Alkyl (C₁₄ 50%; C₁₆ 10%, C₁₂ 40%) | 0.023 | wt % |
| | Dimethyl Benzyl Ammonium Chloride | | |

| | | |
|---|---|---|
| 2-Propanol | 4.950 | wt % |
| Dye | 0.000015 | wt % |
| Distilled Water | 80.067985 | wt % |

Properties Of Example 4

The pH of the moistening fluid is 6.3.

The conductivity of the moistening fluid is 6.7 mmhos

The surface tension of the moistening fluid is 30.4 dynes/cm.

Zone of Inhibition 5.0 mm with a 16 mm saturated disc at 24 hours.

Zone of Inhibition 5.0 mm with a 16 mm saturated disc at 48 hours.

Zone of Inhibition 5.0 mm with a 16 mm saturated disc at 72 hours.

Gross Weight Gain—Sealing Test=0.915 g

Wicking Test Time—2.96 secs.

| | |
|---|---|
| Measurement No. 1: | 1.10 seconds |
| Measurement No. 2: | 0.74 seconds |
| Measurement No. 3: | 0.60 seconds |
| Average Measurement: | 0.81 seconds |

Viscosity—1.34 cps.

### Challenge Test

Log Reductions

| Organism | Zero Time | 24 Hours | 48 Hours | 72 Hours | 7 Days |
|---|---|---|---|---|---|
| Caulobacter | >3.7 | >3.7 | >3.7 | >3.7 | >4.7 |
| Acinetobacter | >3.8 | >3.8 | >3.8 | >3.8 | >4.8 |
| Penicillium | >3.5 | >3.5 | >3.5 | >3.5 | >4.5 |
| Cladosporium | >3.7 | >3.7 | >3.7 | >3.7 | >4.7 |
| Geotrichum | >3.5 | >3.5 | >3.5 | >3.5 | >4.5 |
| Algae | — | >4.3 | >4.3 | >4.3 | >4.3 |
| Ecoli | >3.1 | >3.1 | >3.1 | >3.1 | >4.1 |
| Pseudomonas | >3.5 | >3.5 | >3.5 | >3.5 | >4.4 |
| Candida | 2.5 | >3.2 | >3.2 | >3.2 | >4.2 |
| Aspergillus | 0.96 | >3.0 | >3.0 | >3.0 | >4.0 |
| Sacchromyces | >3 | >3.0 | >3.0 | >3.0 | >4.0 |
| cereviseae | | | | | |

Performance of Example 4
1. The moistening fluid's biocidal capability was at an acceptable level with no regrowth visible in the zone of inhibition.
2. The moistening fluid's conductivity was sufficiently high to be acceptable for use in mailing systems with ion detecting moistening fluids sensors.
3. The results of the sealing test were satisfactory, in that all the envelopes sealed.
4. The viscosity, surface tension and pH were within an acceptable range.
5. The challenge test results indicated acceptable immediate and sustained microbial log reduction of target organisms.

### EXAMPLE 5

Composition

| Detergent (Alkylether hydroxypropyl sultaine) | | 14.950 | wt % |
|---|---|---|---|
| Biocide | Alkyl (C₁₄ 50%; C₁₆ 10%, C₁₂ 40%) | 0.023 | wt % |
| | Dimethyl Benzyl Ammonium Chloride | | |

| | | |
|---|---|---|
| 2-Propanol | 3.860 | wt % |
| Dye | 0.000015 | wt % |
| Distilled Water | 81.166985 | wt % |

Properties of Example 5

The pH of the moistening fluid is 6.2.

The conductivity of the moistening fluid is 6.58 mmhos

The surface tension of the moistening fluid is 33.0 dynes/cm.

Zone of inhibition 5.0 mm with a 16 mm saturated disc at 24 hours.

Zone of inhibition 5.0 mm with a 16 mm saturated disc at 48 hours.

Zone of inhibition 5.0 mm with a 16 mm saturated disc at 72 hours.

Gross Weight Gain—Sealing Test 0.915 g.

Wicking Test Times

| | |
|---|---|
| Measurement No. 1: | 1.20 seconds |
| Measurement No. 2: | 1.60 seconds |
| Measurement No. 3: | 1.59 seconds |
| Average Measurement: | 1.46 seconds |

Viscosity—1.44 cps.

Performance of Example 5
1. The moistening fluid's biocidal capability was at an acceptable level with no regrowth visible in the zone of inhibition.
2. The moistening fluid's conductivity was sufficiently high to be acceptable for use in mailing systems with ion detecting moistening fluids sensors.
3. The results of the sealing test were satisfactory, in that all the envelopes sealed.
4. The viscosity, surface tension and pH were within an acceptable range.

### EXAMPLE 6

Composition

| Detergent (Alkylether hydroxypropyl sultaine) | | 14.950 | wt % |
|---|---|---|---|
| Biocide | Alkyl (C₁₄ 50%; C₁₆ 10%, C₁₂ 40%) | 0.023 | wt % |
| | Dimethyl Benzyl Ammonium Chloride | | |

| | | |
|---|---|---|
| 2-Propanol | 6.220 | wt % |
| Dye | 0.000015 | wt % |
| Distilled Water | 78.806985 | wt % |

Properties of Example 6

The pH of the moistening fluid is 6.3.

The conductivity of the moistening fluid is 6.31 mmhos.

The surface tension of the moistening fluid is 35.1 dynes/cm.

Zone of inhibition 6.0 mm with a 16 mm saturated disc at 24 hours.

Zone of inhibition 6.0 mm with a 16 mm saturated disc at 48 hours.

Zone of inhibition 6.0 mm with a 16 mm saturated disc at 72 hours.

Gross Weight Gain—Sealing Test=0.850 g.

Wicking Test Times

| | |
|---|---|
| Measurement No. 1: | 0.91 seconds |
| Measurement No. 2: | 1.20 seconds |
| Measurement No. 3: | 0.90 seconds |
| Average Measurement: | 1.00 seconds |

Viscosity—1.62 cps.

Performance of Example 6
1. The moistening fluid's biocidal capability was at an acceptable level with no regrowth visible in the zone of inhibition.
2. The moistening fluid's conductivity was sufficiently high to be acceptable for use in mailing systems with ion detecting moistening fluids sensors.
3. The results of the sealing test were satisfactory, in that all the envelopes sealed.
4. The viscosity, surface tension and pH were within an acceptable range.

### EXAMPLE 7

Omposition

| Detergent (Alkylether hydroxypropyl sultaine) | | 5.750 | wt % |
|---|---|---|---|
| Biocide | Alkyl (C₁₄ 50%; C₁₆ 10%, C₁₂ 40%) | 0.005 | wt % |
| | Dimethyl Benzyl Ammonium Chloride | | |

| | | |
|---|---|---|
| Ethanol denatured | 3.920 | wt % |
| Dye | 0.000 | wt % |
| Distilled Water | 90.325 | wt % |

Properties of Example 7

The pH of the moistening fluid is 6.4.

The conductivity of the moistening fluid is 3.0 mmhos.

The surface tension of the moistening fluid is 31.6 dynes/cm.

Zone of inhibition 0.0 mm with a 45 mm saturated disc at 24 hours.

Zone of inhibition 0.0 mm with a 45 mm saturated disc at 48 hours.

Zone of inhibition 0.0 mm with a 45 mm saturated disc at 72 hours.

Gross Weight Gain—Sealing Test 1.345 g.

Wicking Test Times

| | |
|---|---|
| Measurement No. 1: | 0.35 seconds |
| Measurement No. 2: | 0.65 seconds |
| Measurement No. 3: | 0.95 seconds |
| Average Measurement: | 0.65 seconds |

Viscosity—1.07 cps.

Performance of Example 7
1. The moistening fluid's biocidal capability was at an unacceptable level with no zone of inhibition.
2. The moistening fluid's conductivity was sufficiently high to be acceptable for use in mailing systems with ion detecting moistening fluids sensors.
3. The results of the sealing test were satisfactory, in that all the envelopes sealed.
4. The viscosity, surface tension and pH were within a acceptable range.

### EXAMPLE 8

Composition

| Detergent (Alkylether hydroxypropyl sultaine) | | 5.750 | wt % |
|---|---|---|---|
| Biocide | Alkyl (C₁₄ 50%; C₁₆ 10%, C₁₂ 40%) | 0.011 | wt % |
| | Dimethyl Benzyl Ammonium Chloride | | |

| | | |
|---|---|---|
| Ethanol denatured | 3.920 | wt % |
| Dye | 0.000 | wt % |
| Distilled Water | 90.319 | wt % |

Properties of Example 8

The pH of the moistening fluid is 6.4.

The conductivity of the moistening fluid is 2.91 mmhos

The surface tension of the moistening fluid is 32.2 dynes/cm.

Zone of inhibition 0.00 mm with a 45 mm saturated disc at 24 hours.

Zone of inhibition 0.00 mm with a 45 mm saturated disc at 48 hours.

one of inhibition 0.00 mm with a 45 mm saturated disc at 72 hours.

Gross Weight Gain—Sealing Test 0.380 g.

Wicking Test Times

| | |
|---|---|
| Measurement No. 1: | 0.70 seconds |
| Measurement No. 2: | 0.75 seconds |
| Measurement No. 3: | 0.80 seconds |
| Average Measurement: | 0.75 seconds |

Viscosity—1.34 cps.

Performance of Example 8
1. The moistening fluid's biocidal capability was at an unacceptable level with no zone of inhibition.
2. The moistening fluid's conductivity was sufficiently high to be acceptable for use in mailing systems with ion detecting moistening fluids sensors.
3. The results of the sealing test were satisfactory, in that all the envelopes sealed.
4. The viscosity, surface tension and pH were within a acceptable range.

### EXAMPLE 9

Composition

| Detergent (Alkylether hydroxypropyl sultaine) | | 5.750 | wt % |
|---|---|---|---|
| Biocide | Alkyl (C₁₄ 50%; C₁₆ 10%, C₁₂ 40%) | 0.023 | wt % |
| | Dimethyl Benzyl Ammonium Chloride | | |

| | | |
|---|---|---|
| Ethanol denatured | 3.920 | wt % |
| Dye | 0.000 | wt % |
| Distilled Water | 90.307 | wt % |

Properties of Example 9

The pH of the moistening fluid is 6.3.

The conductivity of the moistening fluid is 2.90 mmhos

The surface tension of the moistening fluid is 31.5 dynes/cm.

Zone of Inhibition 0.00 mm with a 45 mm saturated disc at 24 hours.

Zone of Inhibition 0.00 mm with a 45 mm saturated disc at 48 hours.

Zone of Inhibition 0.00 mm with a 45 mm saturated disc at 72 hours.

Gross Weight Gain—Sealing Test 0.880 g.

Wicking Test Times

| | |
|---|---|
| Measurement No. 1: | 0.84 seconds |
| Measurement No. 2: | 0.85 seconds |
| Measurement No. 3: | 0.98 seconds |
| Average Measurement: | 0.89 seconds |

Viscosity—1.14 cps.

Performance of Example 9
1. The moistening fluid's biocidal capability was at an unacceptable level with no zone of inhibition.
2. The moistening fluid's conductivity was sufficiently high to be acceptable for use in mailing systems with ion detecting moistening fluids sensors
3. The results of the sealing test were satisfactory, in that all the envelopes sealed.
4. The viscosity, surface tension and pH were within a acceptable range.

### EXAMPLE 10

Composition

| Detergent (Alkylether hydroxypropyl sultaine) | | 14.950 | wt % |
|---|---|---|---|
| Biocide | Alkyl (C₁₄ 50%; C₁₆ 10%, C₁₂ 40%) | 0.054 | wt % |
| | Dimethyl Benzyl Ammonium Chloride | | |

| | | |
|---|---|---|
| Ethanol denatured | 6.230 | wt % |
| Dye | 0.000 | wt % |
| Distilled Water | 78.766 | wt % |

Properties of Example 10

The pH of the moistening fluid is 6.2.

The conductivity of the moistening fluid is 5.5 mmhos

The surface tension of the moistening fluid is 32.9 dynes/cm.

Zone of Inhibition 6.0 mm with a 45 mm saturated disc at 24 hours

Zone of Inhibition 6.0 mm with a 45 mm saturated disc at 48 hours

Zone of Inhibition 6.0 mm with 45 mm saturated disc at 72 hours

Gross Weight Gain—Sealing Test 0.785 g.

Wicking Test Times

| | |
|---|---|
| Measurement No. 1: | 1.00 seconds |
| Measurement No. 2: | 0.95 seconds |
| Measurement No. 3: | 1.20 seconds |
| Average Measurement: | 1.05 seconds |

Viscosity—1.54 cps.

Performance of Example 10
1. The moistening fluid's biocidal capability was at an acceptable level with no regrowth visible in the zone of inhibition.
2. The moistening fluid's conductivity was sufficiently high to be acceptable for use in mailing systems with ion detecting moistening fluids sensors.
3. The results of the sealing test were satisfactory, in that all the envelopes sealed.
4. The viscosity, surface tension and pH were within an acceptable range.

### EXAMPLE 11

Composition

| Detergent (Alkylether hydroxypropyl sultaine) | | 14.950 | wt % |
|---|---|---|---|
| Biocide | Alkyl (C₁₄ 50%; C₁₆ 10%, C₁₂ 40%) | 0.023 | wt % |
| | Dimethyl Benzyl Ammonium Chloride | | |

| | | |
|---|---|---|
| Ethanol denatured | 6.230 | wt % |
| Dye | 0.000 | wt % |
| Distilled Water | 78.797 | wt % |

Properties of Example 11

The pH of the moistening fluid is 6.4.

The conductivity of the moistening fluid is 5.4 mmhos.

The surface tension of the moistening fluid is 30.0 dynes/cm.

Zone of Inhibition 15 mm with a 45 mm saturated disc at 24 hours.

Zone of Inhibition 15 mm with a 45 mm saturated disc at 48 hours.

Zone of Inhibition 15 mm with a 45 mm saturated disc at 72 hours.

Gross Weight Gain—Sealing Test 0.915 g.

Wicking Test Times

| | |
|---|---|
| Measurement No. 1: | 0.85 seconds |
| Measurement No. 2: | 1.11 seconds |
| Measurement No. 3: | 1.27 seconds |
| Average Measurement: | 1.08 seconds |

Viscosity—1.12 cps.

Performance of Example 11
1A. The moistening fluid's biocidal capability was at an acceptable level with a zone of inhibition of 15 mm at the 24 hour reading.
1B. At the 48 and 72 hour readings there appeared to be a regrowth within the zone of inhibition.
2. The moistening fluid's conductivity was sufficiently high to be acceptable for use in mailing systems with ion detecting moistening fluids sensors.
3. The results of the sealing test were satisfactory, in that all the envelopes sealed.
4. The viscosity, surface tension and pH were within an acceptable range.

### EXAMPLE 12

Composition

| Detergent (Alkylether hydroxypropyl sultaine) | | 11.500 | wt % |
|---|---|---|---|
| Biocide | Alkyl (C₁₄ 50%; C₁₆ 10%, C₁₂ 40%) | 0.043 | wt % |
| | Dimethyl Benzyl Ammonium Chloride | | |

| | | |
|---|---|---|
| Ethanol denatured | 6.230 | wt % |
| Dye | 0.000 | wt % |
| Distilled Water | 82.227 | wt % |

Properties of Example 12

The pH of the moistening fluid is 5.6.

The conductivity of the moistening fluid is 5.37 mmhos

The surface tension of the moistening fluid is 31.4 dynes/cm.

Zone of Inhibition 6.0 mm with a 16 mm saturated disc at 24 hours.

Zone of Inhibition 5.0 mm with a 16 mm discs at 48 hours.

Zone of Inhibition 4.0 mm with a 16 mm saturated disc at 72 hours.

Gross Weight Gain—Sealing Test 1.285 g.

Wicking Test Times

| | |
|---|---|
| Measurement No. 1: | 0.64 seconds |
| Measurement No. 2: | 1.04 seconds |
| Measurement No. 3: | 0.80 seconds |
| Average Measurement: | 0.83 seconds |

Viscosity—1.70 cps.

Performance of Example 12
1A. The moistening fluid's biocidal capability was at an acceptable level with an initial zone of inhibition at 5.0 mm at the 24 hour reading.
1B. At the 48 and 72 hour readings there appeared to be a regrowth within the zone of inhibition.
2. The moistening fluid's conductivity was sufficiently high to be acceptable for use in mailing systems with ion detecting moistening fluids sensors.
3. The results of the sealing test were satisfactory, in that the envelopes sealed.
4. The viscosity, surface tension and pH were within an acceptable range.

### EXAMPLE 13

Composition

| Detergent (Alkylether hydroxypropyl sultaine) | | 11.500 | wt % |
|---|---|---|---|
| Biocide | Alkyl (C₁₄ 50%; C₁₆ 10%, C₁₂ 40%) | 0.074 | wt % |
| | Dimethyl Benzyl Ammonium Chloride | | |

| | | |
|---|---|---|
| Ethanol denatured | 6.230 | wt % |
| Dye | 0.000 | wt % |
| Distilled Water | 82.196 | wt % |

Properties of Example 13

The pH of the moistening fluid is 5.3.

The conductivity of the moistening fluid is 5.32 mmhos

The surface tension of the moistening fluid is 33.5 dynes/cm.

Zone of Inhibition 6.0 mm with a 16 mm saturated disc at 24 hours.

Zone of Inhibition 5.0 mm with a 16 mm saturated disc at 48 hours.

Zone of Inhibition 4.0 mm with a 16 mm saturated disc at 72 hours.

Gross Weight Gain—Sealing Test 1.255 g.

Wicking Test Times

| | |
|---|---|
| Measurement No. 1: | 1.08 seconds |
| Measurement No. 2: | 0.52 seconds |
| Measurement No. 3: | 1.01 seconds |
| Average Measurement: | 0.87 seconds |

Viscosity—1.60 cps.

Performance of Example 13
1A. The moistening fluid's biocidal capability was at an acceptable level with a zone of inhibition at 6.0 mm at the 24 hour reading.
1B. At the 48 and 72 hour readings there appeared to be a regrowth within the zone of inhibition.
2. The moistening fluid's conductivity was sufficiently high to be acceptable for use in mailing systems with ion detecting moistening fluids sensors.
3. The results of the sealing test were satisfactory, in that all the envelopes sealed.
4. The viscosity, surface tension and pH were within an acceptable range.

### EXAMPLE 14

Composition

| Detergent (Alkylether hydroxypropyl sultaine) | | 8.050 | wt % |
|---|---|---|---|
| Biocide | Alkyl (C₁₄ 50%; C₁₆ 10%, C₁₂ 40%) | 0.023 | wt % |
| | Dimethyl Benzyl Ammonium Chloride | | |

| | | |
|---|---|---|
| n-propanol | 4.950 | wt % |
| Dye | 0.000015 | wt % |
| Distilled Water | 86.976985 | wt % |

Properties of Example 14

The pH of the moistening fluid is 6.2

The conductivity of the moistening fluid is 4.03 mmhos

The surface tension of the moistening fluid is 34.4 dynes/cm.

Zone of Inhibition 7.0 mm with a 16 mm saturated disc at 24 hours.

Zone of Inhibition 9.0 mm with a 16 mm saturated disc at 48 hours.

Zone of Inhibition 9.0 mm with a 16 mm saturated disc at 72 hours.

Gross Weight Gain—Sealing Test 0.700 g.

Wicking Test Times

| | |
|---|---|
| Measurement No. 1: | 0.82 seconds |
| Measurement No. 2: | 0.90 seconds |
| Measurement No. 3: | 0.81 seconds |
| Average Measurement: | 0.84 seconds |

Viscosity—1.32 cps.

Performance of Example 14
1. The moistening fluid's biocidal capability was at an acceptable level with no regrowth visible in the Zone of Inhibition.
2. The moistening fluid's conductivity was sufficiently high to be acceptable for use in mailing systems with ion detecting moistening fluids sensors.
3. The results of the sealing test were satisfactory, in that all the envelopes sealed.
4. The viscosity, surface tension and pH were within an acceptable range.

### EXAMPLE 15

Composition

| Detergent (Alkylether hydroxypropyl | | 8.050 | wt % |
|---|---|---|---|
| sultaine) | | | |
| Biocide | 1,2-Benzisothiazoline-3-one | 0.057 | wt % |

| | | |
|---|---|---|
| 2-Propanol | 4.950 | wt % |
| Dye | 0.000015 | wt % |
| Distilled Water | 86.942985 | wt % |

Properties of Example 15

The pH of the moistening fluid is 6.4.

The conductivity of the moistening fluid is 3.63 mmhos

The surface tension of this moistening fluid is 33.3 dynes/cm

Zone of inhibition 1.0 mm with a 16 mm saturated disc at 24 hours.

Zone of inhibition 1.0 mm with a 16 mm saturated disc at 48 hours.

Zone of inhibition 1.0 mm with a 16 mm saturated disc at 72 hours.

Gross Weight Gain—Sealing Test 0.685 g.

Wicking Test Times

| | |
|---|---|
| Measurement No. 1: | 0.60 seconds |
| Measurement No. 2: | 0.80 seconds |
| Measurement No. 3: | 1.00 seconds |
| Average Measurement: | 0.80 seconds |

Viscosity—1.34 cps.

Performance of Example 15
1. The moistening fluid's biocidal capability was at an acceptable level with no regrowth visible in the zone of inhibition.
2. The moistening fluid's conductivity was sufficiently high to be acceptable for use in mailing systems with ion detecting moistening fluids sensors.
3. The results of the sealing test were satisfactory, in that all the envelopes sealed.
4. The viscosity, surface tension and pH were within an acceptable range.

### EXAMPLE 16

Composition

| Detergent (Alkylether hydroxypropyl sultaine) | | 8.050 | wt % |
|---|---|---|---|
| Biocide | (Glycine, N-(hydroxymethyl)-monosodium | 0.100 | wt % |
| | salt) | | |

| | | |
|---|---|---|
| n-Propanol | 4.950 | wt % |
| Dye | 0.000015 | wt % |
| Distilled Water | 86.899985 | wt % |

Properties of Example 16

The pH of the moistening fluid is 6.8.

The conductivity of the moistening fluid is 3.93 mmhos

The surface tension of this moistening fluid is 32.5 dynes/cm

Zone of inhibition 2.0 mm with a 16 mm saturated disc at 24 hours.

Zone of inhibition 2.0 mm with a 16 mm saturated disc at 48 hours.

Zone of inhibition 2.0 mm with a 16 mm saturated disc at 72 hours.

Gross Weight Gain—Sealing Test 0.820 g.

Wicking Test Times

| | |
|---|---|
| Measurement No. 1: | 1.83 seconds |
| Measurement No. 2: | 1.02 seconds |
| Measurement No. 3: | 1.36 seconds |
| Average Measurement: | 1.40 seconds |

Viscosity—1.29 cps.

Performance of Example 16
1. The moistening fluid's biocidal capability was at an acceptable level with no regrowth visible in the zone of inhibition.
2. The moistening fluid's conductivity was sufficiently high to be acceptable for use in mailing systems with ion detecting moistening fluids sensors.
3. The results of the sealing test were satisfactory, in that all the envelopes sealed.
4. The viscosity, surface tension and pH were within an acceptable range.

### EXAMPLE 17

Composition

| Detergent (Alkylether hydroxypropyl sultaine) | | 8.050 | wt % |
|---|---|---|---|
| Biocide | 5-Chloro-2-Methyll-4-Isothioazolin-3-one | 0.016 | wt % |

| | | |
|---|---|---|
| 2-Propanol | 4.950 | wt % |
| Dye | 0.000015 | wt % |
| Distilled Water | 86.983985 | wt % |

Properties of Example 17

The pH of the moistening fluid is 6.3.

The conductivity of the moistening fluid is 3.81 mmhos

The surface tension of this moistening fluid is 32.8 dynes/cm

Zone of inhibition 2.5 mm with a 16 mm saturated disc at 24 hours.

Zone of inhibition 2.5 mm with a 16 mm saturated disc at 48 hours.

Zone of inhibition 2.5 mm with a 16 mm saturated disc at 72 hours.

Gross Weight Gain—Sealing Test 0.735 g.

Wicking Test Times

| | |
|---|---|
| Measurement No. 1: | 0.77 seconds |
| Measurement No. 2: | 0.83 seconds |
| Measurement No. 3: | 1.20 seconds |
| Average Measurement: | 0.93 seconds |

Viscosity—1.25 cps.

Performance of Example 17
1. The moistening fluid's biocidal capability was at an acceptable level with no regrowth visible in the zone of inhibition.
2. The moistening fluid's conductivity was sufficiently high to be acceptable for use in mailing systems with ion detecting moistening fluids sensors.
3. The results of the sealing test were satisfactory, in that all the envelopes sealed.
4. The viscosity, surface tension and pH were within an acceptable range.

### EXAMPLE 18

### 100% Distilled Water

Properties of Example 18

The pH of the moistening fluid is 6.7.

The conductivity of the moistening fluid is 10.4 micromhos

The surface tension of this moistening fluid is 67.4 dynes/cm

Zone of inhibition 0.0 mm with a 16 mm saturated disc at 24 hours.

Zone of inhibition 0.0 mm with a 16 mm saturated disc at 48 hours.

Zone of inhibition 0.0 mm with a 16 mm saturated disc at 72 hours.

Gross Weight Gain—Sealing Test=0.704 g.

Wicking Test Times

| | |
|---|---|
| Measurement No. 1: | 2.15 seconds |
| Measurement No. 2: | 1.43 seconds |
| Measurement No. 3: | 2.22 seconds |
| Average Measurement: | 1.93 seconds |

Viscosity—0.89 cps

Performance of Example 18
1. The moistening fluid's biocidal capability was not acceptable, as there was no zone of inhibition.
2. The moistening fluid's conductivity was not sufficiently high to be acceptable for use in mailing systems with ion detecting moistening fluids sensors.
3. The results of the sealing test were satisfactory, in that all the envelopes sealed.

### EXAMPLE 19

### 100% Deionized Water

Properties of Example 19

The pH of the moistening fluid is 6.7.

The conductivity of the moistening fluid is 175 micromohs

The surface tension of this moistening fluid is 59.5 dynes/cm

Zone of inhibition 0.0 mm with a 16 mm saturated disc at 24 hours.

Zone of inhibition 0.0 mm with a 16 mm saturated disc at 48 hours.

Zone of inhibition 0.0 mm with a 16 mm saturated disc at 72 hours.

Gross Weight Gain—Sealing Test 0.532 g.

Wicking Test Times

| | |
|---|---|
| Measurement No. 1: | 1.61 seconds |
| Measurement No. 2: | 1.35 seconds |
| Measurement No. 3: | 1.84 seconds |
| Average Measurement: | 1.60 seconds |

Viscosity—0.89 cps.

Performance of Example 19
1. The moistening fluid's biocidal capability was not acceptable as there was no zone of inhibition.
2. The moistening fluid's conductivity was not sufficiently high to be acceptable for use in mailing systems with ion detecting moistening fluids sensors.
3. The results of the sealing test were satisfactory, in that all the envelopes sealed.

### EXAMPLE 20

### 100% Tap Water

Properties of Example 20

The pH of the moistening fluid is 6.3.

The conductivity of the moistening fluid is 167.3 micromohs

The surface tension of this moistening fluid is 58.1 dynes/cm

Zone of inhibition 0.0 mm with a 16 mm saturated disc at 24 hours.

Zone of inhibition 0.0 mm with a 16 mm saturated disc at 48 hours.

Zone of inhibition 0.0 mm with a 16 mm saturated disc at 72 hours.

Gross Weight Gain—Sealing Test 0. g.

Wicking Test Time

| | |
|---|---|
| Measurement No. 1: | 3.30 seconds |
| Measurement No. 2: | 3.56 seconds |
| Measurement No. 3: | 3.71 seconds |
| Average Measurement: | 3.52 seconds |

Viscosity—1.00 cps.

Challenge Test

Log Reductions

| Organism | Zero Time | 24 Hours | 48 Hours | 72 Hours | 7 Days |
|---|---|---|---|---|---|
| Caulobacter | 0 | 0 | 0 | 0 | 0 |
| Pseudomonas | 0 | 0 | 0 | 0 | 0 |
| Candida | 0 | 0 | 0 | 0 | 0 |
| Aspergillus | 0 | 0 | 0 | 0 | 0 |
| Sacchromyces | 0 | 0 | 0 | 0 | 0 |
| cereviseae | | | | | |

Performance of Example 20
1. The moistening fluid's biocidal capability was not acceptable as there was no zone of inhibition.
2. The moistening fluid's conductivity was not sufficiently high to be acceptable for use in mailing systems with ion detecting moistening fluids sensors.
3. The results of the sealing test were satisfactory, in that all the envelopes sealed.
4. The challenge test results indicated no log reduction of target organisms and therefore were unacceptable.

### EXAMPLE 21

Composition

| Detergent (Alkylether hydroxypropyl sultaine) | | 8.050 | wt % |
|---|---|---|---|
| Biocide | Alkyl (C₁₄ 50%; C₁₆ 10%, C₁₂ 40%) | 0.023 | wt % |
| | Dimethyl Benzyl Ammonium Chloride | | |

| | | |
|---|---|---|
| 1-Butanol | 4.950 | wt % |
| Dye | 0.000015 | wt % |
| Distilled Water | 86.976985 | wt % |

Properties of Example 21

The pH of the moistening fluid is 6.4.

The conductivity of the moistening fluid is 3.90 mmhos

The surface tension of this moistening fluid is 28.5 dynes/cm

Zone of inhibition 4.0 mm with a 16 mm saturated disc at 24 hours.

Zone of inhibition 4.0 mm with a 16 mm saturated disc at 48 hours.

Zone of inhibition 4.0 mm with a 16 mm saturated disc at 72 hours.

Gross Weight Gain—Sealing Test 1.0 g.

Wicking Test Times

| | | |
|---|---|---|
| Measurement No. 1: | 0 minutes | 0.75 seconds |
| Measurement No. 2: | 0 minutes | 1.00 seconds |
| Measurement No. 3: | 0 minutes | 0.82 seconds |
| Average Measurement: | 0 minutes | 0.86 seconds |

Viscosity—1.24 cps.

Performance of Example 21
1. The moistening fluid's biocidal capability was at an acceptable level with no regrowth visible in the zone of inhibition.
2. The moistening fluid's conductivity was sufficiently high to be acceptable for use in mailing systems with ion detecting moistening fluids sensors.
3. The results of the sealing test were satisfactory, in that all the envelopes sealed.
4. The viscosity, surface tension and pH were within an acceptable range.

Normal public drinking waters, i.e., tap water and well water, are often thought to be an adequate substitute for a moistening solution. There are many allowable, regulated contaminants found in most public drinking waters. Contaminants, such as coliform bacteria, lead, copper, calcium and chlorine, even if present under the maximum allowable contaminant levels, over time, could have an adverse effect on moistening systems. Therefore, it is unadvisable to use normal public drinking waters as an alternative to moistening fluids. It was also found that distilled and de-ionized water provide no protection, as a moistening fluid, from random environmental growth, which commonly contaminate stagnant waters, thereby making each an unacceptable substitute for a moistening fluid.

[image]

Zone of Inhibition Testing with Examples 2 and 4 with Water Controls

Zone of Inhibition with 16 mm disc
Average of Plates

Incubation
Time in Hours

| Formula | 0 | 24 | 48 | 72 |
|---|---|---|---|---|
| Example 2 (mm) | | 4.75 | 5.0 | 5.0 |
| Example 4 (mm) | | 5 | 5 | 5 |
| Deionized Water | | 0 | 0 | 0 |
| (mm) | | | | |
| Distilled Water (mm) | | 0 | 0 | 0 |
| Tap Water (mm) | | 0 | 0 | 0 |

Examples 2 and 4 were tested with normal public drinking waters, from Shelton, Conn. and distilled and deionized water. Examples 2 and 4 showed a zone of inhibition of 5.0 mm at 72 hours. When normal public drinking waters, from Shelton, Conn. or deionized or distilled water were used alone, they showed no ability to kill the targeted micro-organisms or inhibit bacteria or fungi growth.

The above embodiments have been given by way of illustration only, and other embodiments of the instant invention will be apparent to those skilled in the art, from consideration of the detailed description. Accordingly, any limitation on the instant invention is to be found only in the claims.

What is claimed is:

1. A system having a moistening device, the improvement comprising: within the moistening device employing a fluid containing detergent, biocide, alcohol, and water that inhibits and/or destroys the growth of specific bacteria, fungi and algae.

2. The system claimed in claim 1, wherein the fluid is inert to plastic materials contained in moistening devices.

3. The system claimed in claim 1, wherein the fluid further includes a dye.

4. The system claimed in claim 1, wherein the fluid composition consists essentially of:
5.75-14.95 (wt %) Detergent;
0.005-0.107 (wt %) Biocide;
3.86-6.22 (wt %) Alcohol;
0.00-0.000015 (wt %) Dye; and
78.77-90.33 (wt %) Distilled Water.

5. The composition claimed in claim 4, wherein the detergent is Alkylether hydroxypropyl sultaine.

6. The composition claimed in claim 4, wherein the Biocides are selected from the group consisting of: Alkyl (C₁₄ 50%; C₁₆ 10%, C₁₂ 40) Dimethyl Benzyl Ammonium Chloride; 5-Chloro-2-Methyl-4-Isothioazolin-3-one; N-(hydroxymethyl)-monosodium salt; and 1,2-Benzisothiazoline-3-one.

7. The composition claimed in claim 4, wherein the Alcohols are selected from the group consisting of: 2-Propanol; Ethanol (denatured); n-Propanol; and 1-Butanol.

8. The composition claimed in claim 4, wherein the dye is Food Drug & Cosmetic Blue Dye #1.

9. The system claimed in claim 1, wherein the pH of the fluid is between 5.3 and 6.8.

10. The system claimed in claim 1, wherein the surface tension of the fluid is between 30.0 dynes/cm and 67.4 dynes/cm.

11. The system claimed in claim 1, wherein the conductivity of the fluid is between 10.4 micromohs and 6.7 millimohs.

12. The system claimed in claim 1, wherein the viscosity of the fluid is between 0.89 cps.-1.70 cps.

13. The system claimed in claim 1, wherein the fluid may be used to seal water moistening adhesive envelopes.

14. The system claimed in claim 1, wherein the fluid may be used to inhibit and/or kill:
A. Sphingomonas paucimobilis (bacteria),
B. Pseudomonas aeruginosa (bacteria),
C. Pseudomonas stutzeri (bacteria),
D. Acinetobacter species (bacteria),
E. Caulobacter species (bacteria),
F. Pseudomonas fluorescens (bacteria),
G. Brevendimonas species (bacteria),
H. Flavomonas species (bacteria), and
I. Escherichia coli (bacteria).

15. The system claimed in claim 1, wherein the fluid may be used to inhibit and/or kill:
A. Geotrichum species (fungus)
B. Cladosporium species (fungus), and
C. Oididendron species (fungus)

16. The system claimed in claim 1, wherein the fluid may be used to inhibit and/or kill: yeasts.

17. The system claimed in claim 1, wherein the fluid may be used to inhibit and/or kill:
Fusarium species (mold),
Aspergillus niger (mold); and
Penicillium species (mold).

18. The system claimed in claim 1, wherein the fluid may be used to inhibit and/or kill: Blue Green algae, Freshwater algae

19. The system claimed in claim 1, wherein the system is a mailing system.

20. The system claimed in claim 1, wherein the moistening device is moistening dispenser.

21. A moistening fluid that inhibits and/or destroys the growth of specific bacteria, fungi and algae consisting essentially of:
5.75-14.95 (wt %) Detergent;
0.005-0.107 (wt %) Biocide;
3.86-6.22 (wt %) Alcohol;
0.00-0.0000015 (wt %) Dye; and
78.77-90.33 (wt %) Distilled Water.

22. The composition claimed in claim 21, wherein the detergent is Alkylether hydroxypropyl sultaine.

23. The composition claimed in claim 21, wherein the Biocides are selected from the group consisting of: Alkyl (C₁₄ 50%; C₁₆ 10%, C₁₂ 40) Dimethyl Benzyl Ammonium Chloride; 5-Chloro-2-Methyl-4-Isothioazolin-3-one; N-(hydroxymethyl)-monosodium salt; and 1,2-Benzisothiazoline-3-one.

24. The composition claimed in claim 21, wherein the Alcohols are selected from the group consisting of: 2-Propanol; Ethanol (denatured); n-Propanol; and 1-Butanol.

25. The composition claimed in claim 21, wherein the dye is Food Drug & Cosmetic Blue Dye #1.

26. The system claimed in claim 21, wherein the pH of the fluid is between 5.3 and 6.8.

27. The system claimed in claim 21, wherein the surface tension of the fluid is between 30.0 dynes/cm and 67.4 dynes/cm.

28. The system claimed in claim 21, wherein the conductivity of the fluid is between 10.4 micromohs and 3.81 millimohs.

29. The system claimed in claim 21, wherein the viscosity of the fluid is between 0.89 cps.-1.70 cps.

30. The system claimed in claim 21, wherein the fluid may be used to seal water moistening adhesive envelopes.

31. The system claimed in claim 21, wherein the fluid may be used to inhibit and/or kill:
A. Sphingomonas paucimobilis (bacteria),
B. Pseudomonas aeruginosa (bacteria),
C. Pseudomonas stutzeri (bacteria),
D. Acinetobacter species (bacteria),
E. Caulobacter species (bacteria),
F. Pseudomonas fluorescens (bacteria),
G. Brevendimonas species (bacteria),
H. Flavomonas species (bacteria), and
I. Escherichia coli (bacteria).

32. The system claimed in claim 21, wherein the fluid may be used to inhibit and/or kill:
A. Geotrichum species (fungus)
B. Cladosporium species (fungus), and
C. Oididendron species (fungus)

33. The system claimed in claim 21, wherein the fluid may be used to inhibit and/or kill: yeasts.

34. The system claimed in claim 21, wherein the fluid may be used to inhibit and/or kill:
Fusarium species (mold),
Aspergillus niger (mold); and
Penicillium species (mold).

35. The system claimed in claim 21, wherein the fluid may be used to inhibit and/or kill: Blue Green algae, Freshwater algae

36. The system claimed in claim 21, wherein the system is a mailing system.

37. The system claimed in claim 21, wherein the moistening device is moistening dispenser.
